(19) United States
(12) Patent Application Publication
(10) Pub. No.: US 2021/0196410 A1
(43) Pub. Date: Jul. 1, 2021

(54) SPECIMEN COLLECTOR FOR ROBOTIC MEDICAL SYSTEM

(71) Applicant: Auris Health, Inc., Redwood City, CA (US)

(72) Inventors: Jason J. Hsu, Mountain View, CA (US); Janet Zhen Bone, San Mateo, CA (US); Rachel Leigh Chok, Santa Clara, CA (US); Jeffrey William Draper, San Francisco, CA (US)

(21) Appl. No.: 17/130,606

(22) Filed: Dec. 22, 2020

Related U.S. Application Data
(60) Provisional application No. 62/955,050, filed on Dec. 30, 2019.

(51) Int. Cl. A61B 34/30; A61B 17/00; A61B 46/10

(52) U.S. Cl. CPC A61B 34/30; A61B 17/00234; A61B 46/10; A61B 34/37; A61B 2034/301; A61B 2034/302; A61B 2034/303; A61B 2017/00287

(57) ABSTRACT

Specimen collectors configured for use with robotic medical systems are described herein. A robotic medical system can include a medical instrument that can be inserted into a patient to capture a specimen. A robotic manipulator can be engaged with the medical instrument and configured to operate the medical instrument. A sterile barrier can be used to separate a sterile field containing the medical instrument from a non-sterile field containing the robotic manipulator. A specimen collector can include a receptacle portion in the sterile field and configured to receive the specimen when the distal end of the medical instrument is withdrawn from the patient and a connector coupled to the receptacle portion and configured to attach to the robotic medical system. The robotic system can deposit the specimen in the receptacle portion.

### PRIORITY APPLICATION

This application claims priority to U.S. Provisional Application No. 62/955,050, filed Dec. 30, 2019, which is incorporated herein by reference. Any and all applications for which a foreign or domestic priority claim is identified in the Application Data Sheet as filed with the present application are hereby incorporated by reference under 37 CFR 1.57.

### TECHNICAL FIELD

The systems and methods disclosed herein are directed to robotic medical systems, and more particularly to a specimen collector for robotic medical systems.

### BACKGROUND

Robotic medical systems can be configured to perform a wide variety of medical procedures, including endoscopic, laparoscopic and open procedures among others. During some procedures, medical instruments can be used to remove an object, specimen, or sample from a patient. As one example, a medical instrument can be used to remove a kidney stone or kidney stone fragment during a kidney stone removal procedure.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an embodiment of a cart-based robotic system arranged for diagnostic and/or therapeutic bronchoscopy.

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopic procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopic procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates a top view of an embodiment a robotic medical system including a specimen collector.

FIG. 22 is a side view of the robotic medical system and specimen collector of FIG. 21.

FIG. 23 is a perspective view of a distal end of an embodiment of a robotic arm covered with a sterile drape and including a specimen collector.

FIG. 24 is a block diagram illustrating example control components for the robotic medical system of FIG. 21.

FIG. 25 is a flow chart illustrating an example control method that can be executed using the control components of FIG. 24 to operate the robotic medical system of FIG. 21.

FIG. 26 is a front view of an embodiment of a specimen collector.

FIG. 27 is an exploded view of the specimen collector of FIG. 26.

FIG. 28 is a flowchart illustrating an example method for using a robotic medical system to deposit a specimen in a specimen collector.

FIG. 29 illustrates an isometric view of an embodiment of a sterile drape including specimen collectors.

FIG. 30 is a perspective view of an embodiment of the sterile drape of FIG. 29 installed on a cart including three robotic arms.

FIG. 31 illustrates another embodiment of a sterile drape including specimen collectors.

FIG. 32 illustrates an embodiment of a sterile barrier assembly including a specimen collector.

### DETAILED DESCRIPTION

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopic procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart.

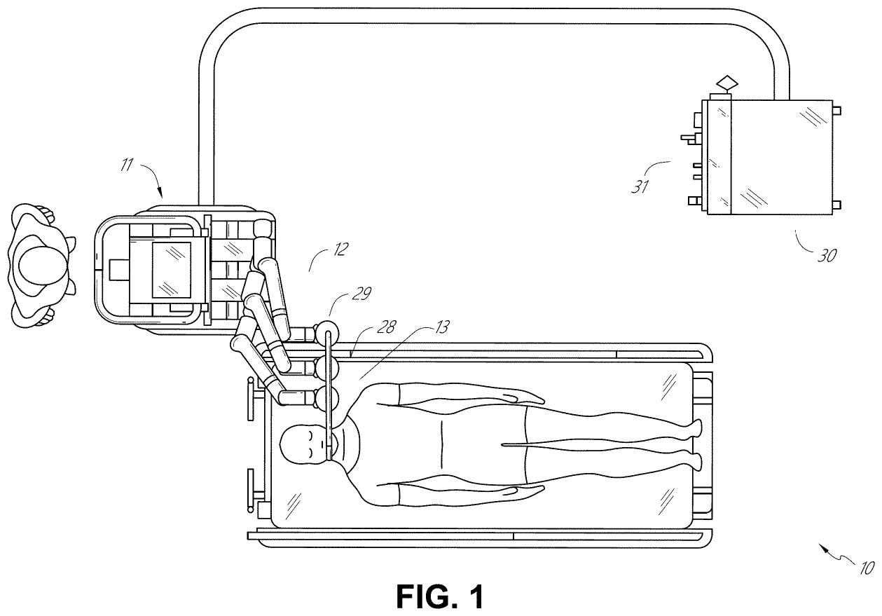

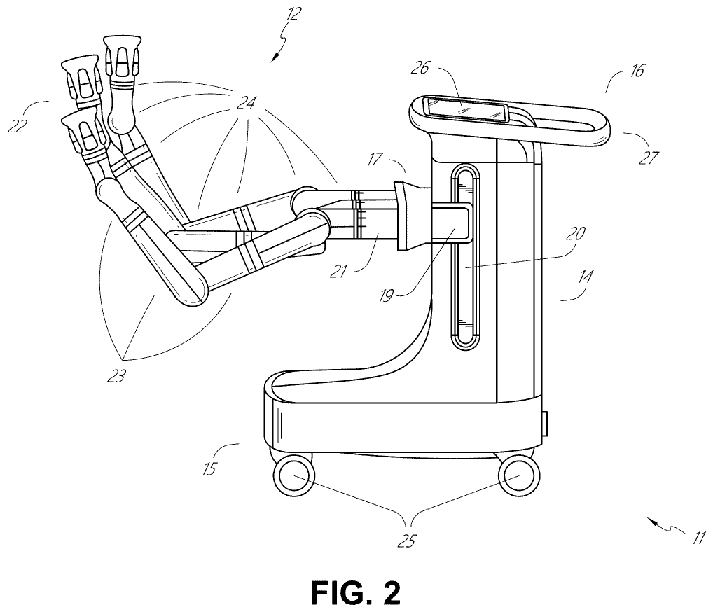

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independently of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or re-positioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of the tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include optoelectronics equipment for detecting, receiving, and processing data received from the optical sensors or cameras throughout the robotic system 10. In combination with the control system, such optoelectronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in the system 10 are generally designed to provide both robotic controls as well as preoperative and real-time information of the procedure, such as navigational and localization information of the endoscope 13. When the console 31 is not the only console available to the physician, it may be used by a second operator, such as a nurse, to monitor the health or vitals of the patient and the operation of the system 10, as well as to provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart 11, the support for controls, optics, fluidics, and/or navigation may be provided through a separate cable.

FIG. 2 provides a detailed illustration of an embodiment of the cart 11 from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage 17 at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of the robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when the carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm 12. Each of the robotic arms 12 may have seven joints, and thus provide seven degrees of freedom. A multitude of joints result in a multitude of degrees of freedom, allowing for “redundant” degrees of freedom. Having redundant degrees of freedom allows the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and robotic arms 12 over the floor. Accordingly, the cart base 15 houses heavier components, such as electronics, motors, power supply, as well as components that either enable movement and/or immobilize the cart 11. For example, the cart base 15 includes rollable wheel-shaped casters 25 that allow for the cart 11 to easily move around the room prior to a procedure. After reaching the appropriate position, the casters 25 may be immobilized using wheel locks to hold the cart 11 in place during the procedure.

Positioned at the vertical end of the column 14, the console 16 allows for both a user interface for receiving user input and a display screen (or a dual-purpose device such as, for example, a touchscreen 26) to provide the physician user with both preoperative and intraoperative data. Potential preoperative data on the touchscreen 26 may include preoperative plans, navigation and mapping data derived from preoperative computerized tomography (CT) scans, and/or notes from preoperative patient interviews. Intraoperative data on display may include optical information provided from the tool, sensor and coordinate information from sensors, as well as vital patient statistics, such as respiration, heart rate, and/or pulse. The console 16 may be positioned and tilted to allow a physician to access the console 16 from the side of the column 14 opposite the carriage 17. From this position, the physician may view the console 16, robotic arms 12, and patient while operating the console 16 from behind the cart 11. As shown, the console 16 also includes a handle 27 to assist with maneuvering and stabilizing the cart 11.

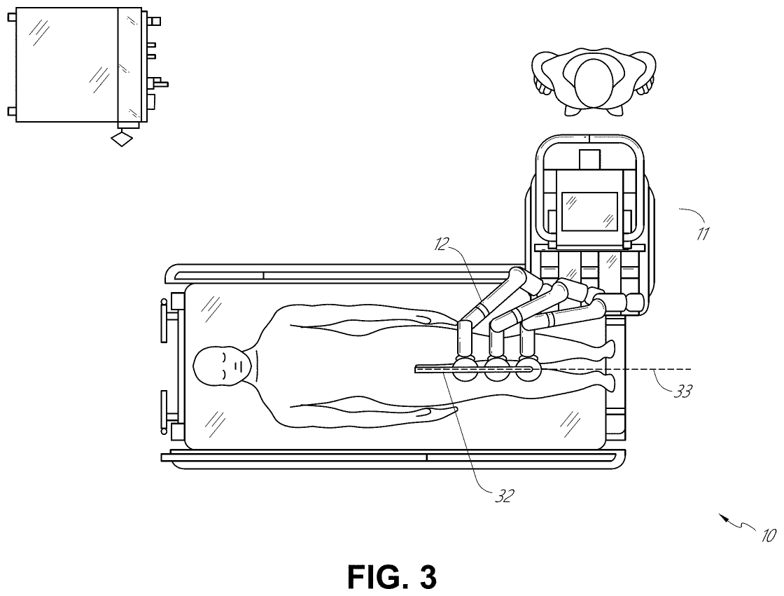

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

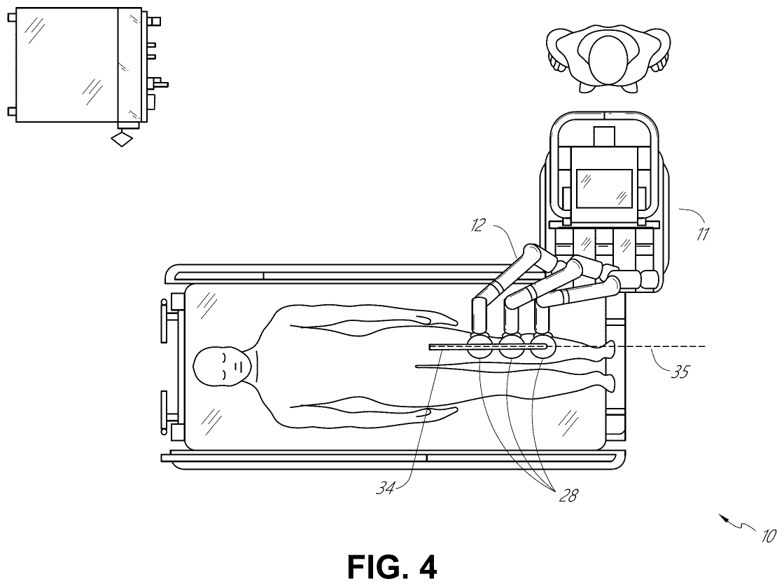

FIG. 4 illustrates an embodiment of a robotically-enabled system 10 similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

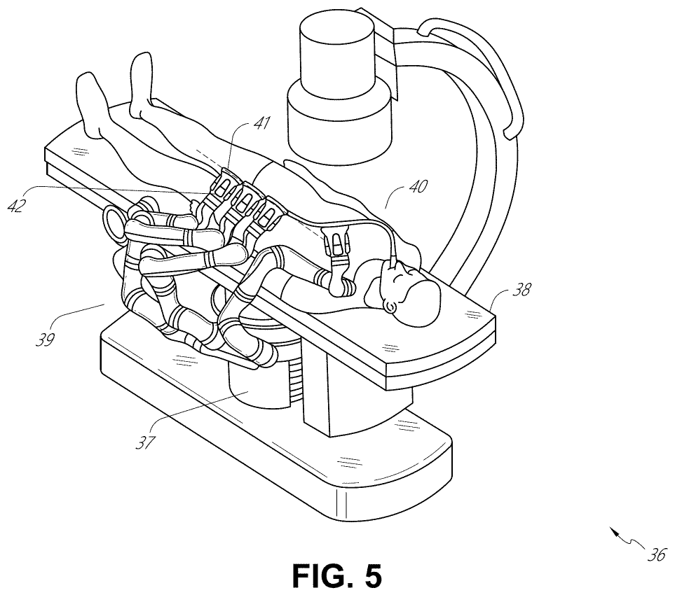

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopic procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around the table 38.

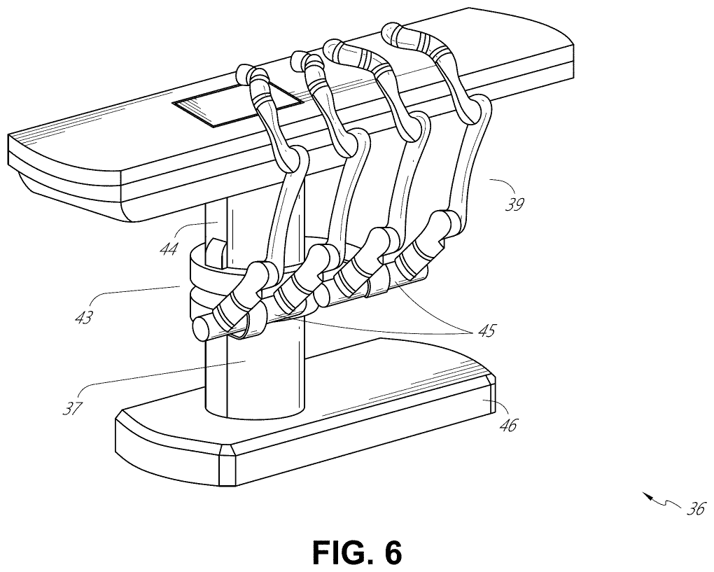

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independently of the other carriages. While the carriages 43 need not surround the column 37 or even be circular, the ring-shape as shown facilitates rotation of the carriages 43 around the column 37 while maintaining structural balance. Rotation and translation of the carriages 43 allows the system 36 to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

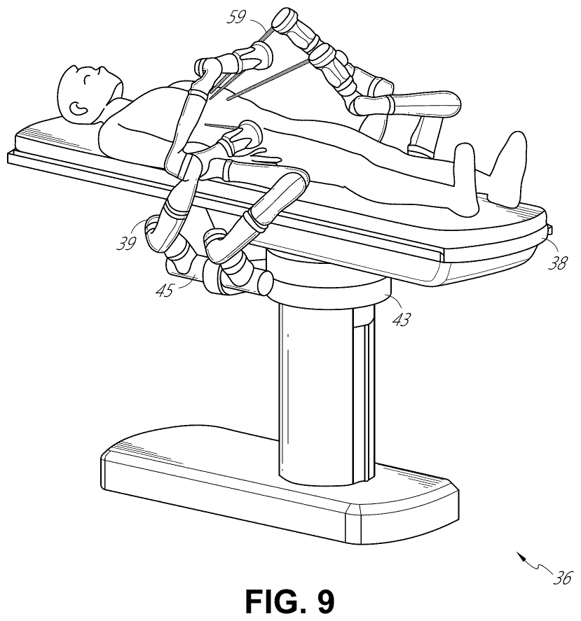

The robotic arms 39 may be mounted on the carriages 43 through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of the table 38 (as shown in FIG. 6), on opposite sides of the table 38 (as shown in FIG. 9), or on adjacent sides of the table 38 (not shown).

The column 37 structurally provides support for the table 38, and a path for vertical translation of the carriages 43. Internally, the column 37 may be equipped with lead screws for guiding vertical translation of the carriages, and motors to mechanize the translation of the carriages 43 based the lead screws. The column 37 may also convey power and control signals to the carriages 43 and the robotic arms 39 mounted thereon.

The table base 46 serves a similar function as the cart base 15 in the cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

With continued reference to FIG. 6, the system 36 may also include a tower (not shown) that divides the functionality of the system 36 between the table and the tower to reduce the form factor and bulk of the table. As in earlier disclosed embodiments, the tower may provide a variety of support functionalities to the table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base 46 for potential stowage of the robotic arms 39. The tower may also include a master controller or console that provides both a user interface for user input, such as keyboard and/or pendant, as well as a display screen (or touchscreen) for preoperative and intraoperative information, such as real-time imaging, navigation, and tracking information. In some embodiments, the tower may also contain holders for gas tanks to be used for insufflation.

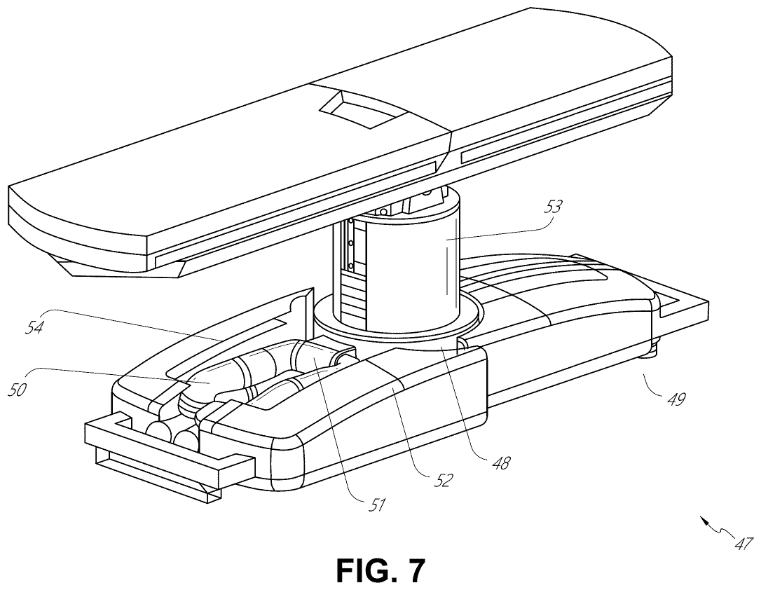

In some embodiments, a table base may stow and store the robotic arms when not in use. FIG. 7 illustrates a system 47 that stows robotic arms in an embodiment of the table-based system. In the system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and robotic arms 50 around column 53, and closed to stow to protect them when not in use. The base covers 52 may be sealed with a membrane 54 along the edges of its opening to prevent dirt and fluid ingress when closed.

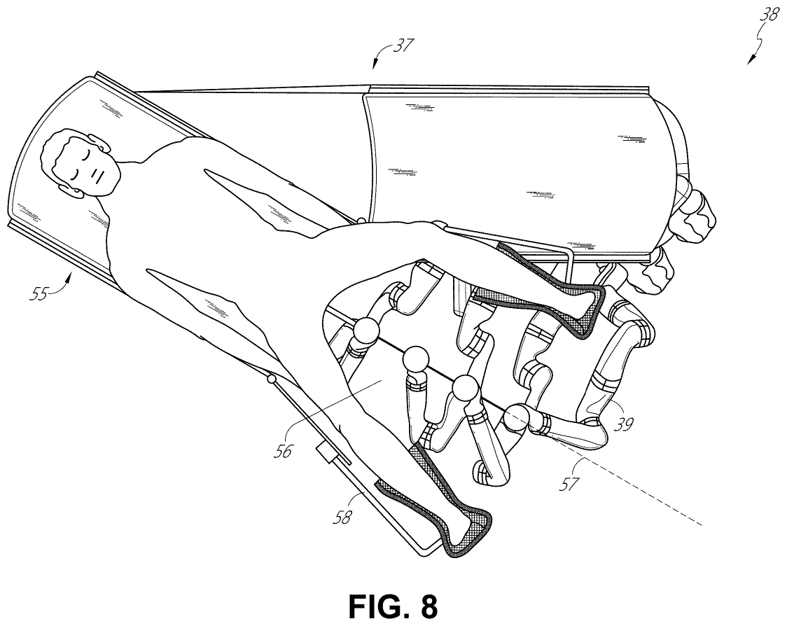

FIG. 8 illustrates an embodiment of a robotically-enabled table-based system configured for a ureteroscopic procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instrument 59 may be positioned using the arm mounts 45 to be passed through minimal incisions on both sides of the patient to reach his/her abdominal cavity.

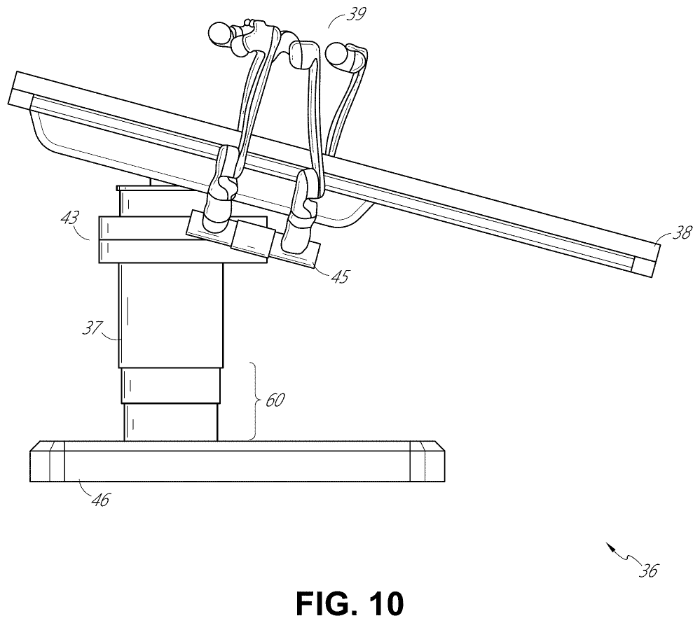

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the robotic arms 39 maintain the same planar relationship with the table 38. To accommodate steeper angles, the column 37 may also include telescoping portions 60 that allow vertical extension of the column 37 to keep the table 38 from touching the floor or colliding with the table base 46.

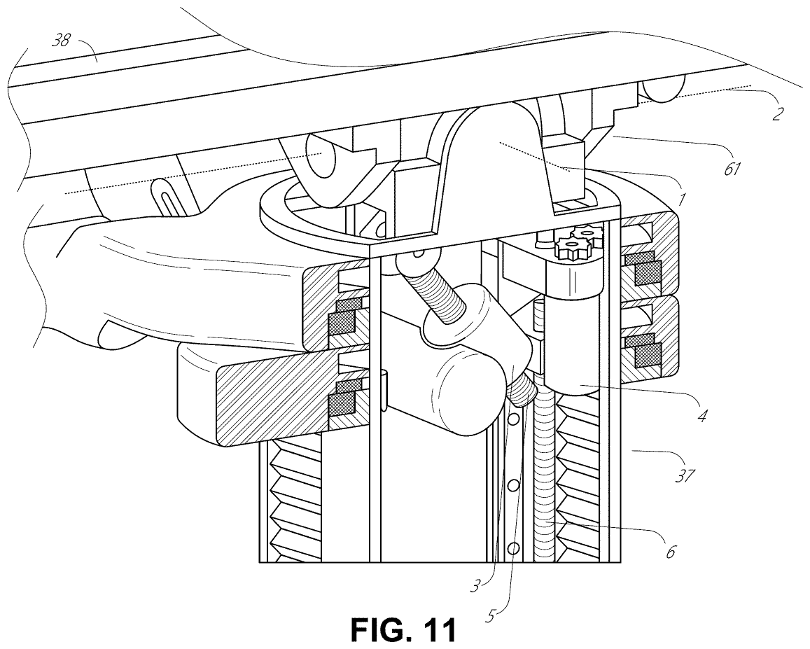

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's upper abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

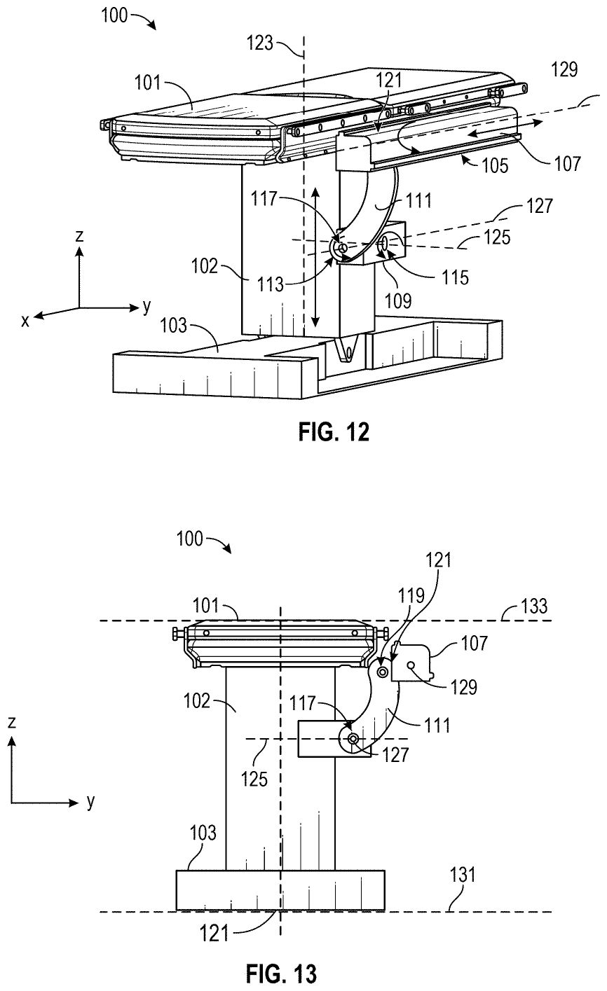

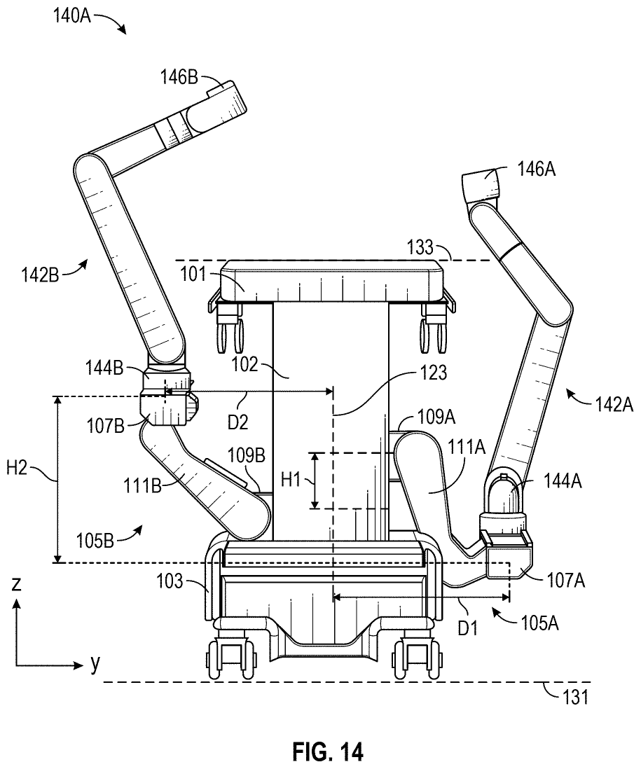

FIGS. 12 and 13 illustrate isometric and end views of an alternative embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (Z-lift) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms may comprise (i) an instrument driver (alternatively referred to as “instrument drive mechanism” or “instrument device manipulator”) that incorporates electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

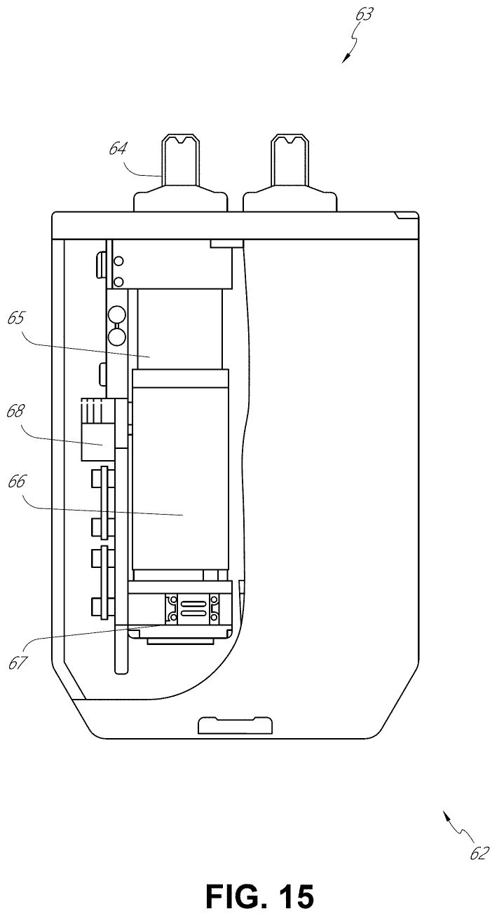

FIG. 15 illustrates an example instrument driver. Positioned at the distal end of a robotic arm, instrument driver 62 comprises one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independently controlled and motorized, the instrument driver 62 may provide multiple (e.g., four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

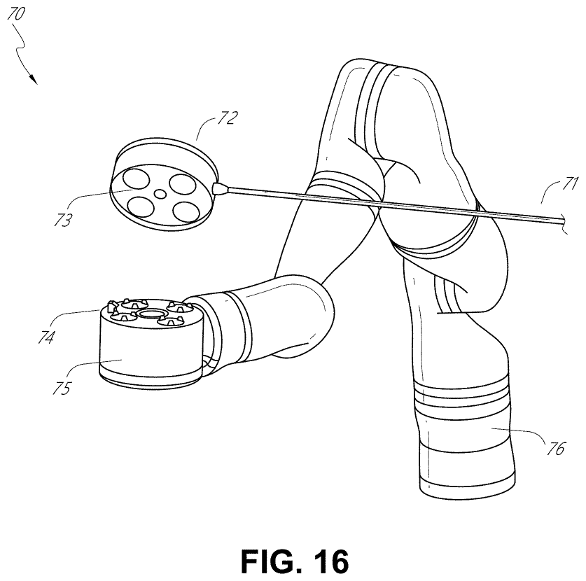

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of the instrument base 72 may share axes of rotation with the drive outputs 74 in the instrument driver 75 to allow the transfer of torque from the drive outputs 74 to the drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the elongated shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at the distal end of the elongated shaft 71, where tension from the tendon causes the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on the drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing therebetween may be altered or engineered for specific purposes, wherein tighter spiraling exhibits lesser shaft compression under load forces, while lower amounts of spiraling results in greater shaft compression under load forces, but limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft 71 may comprise a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include an optical camera. The shaft 71 may also accommodate optical fibers to carry light from proximally-located light sources, such as light emitting diodes, to the distal end of the shaft 71.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft 71. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft 71 during an endoscopic procedure.

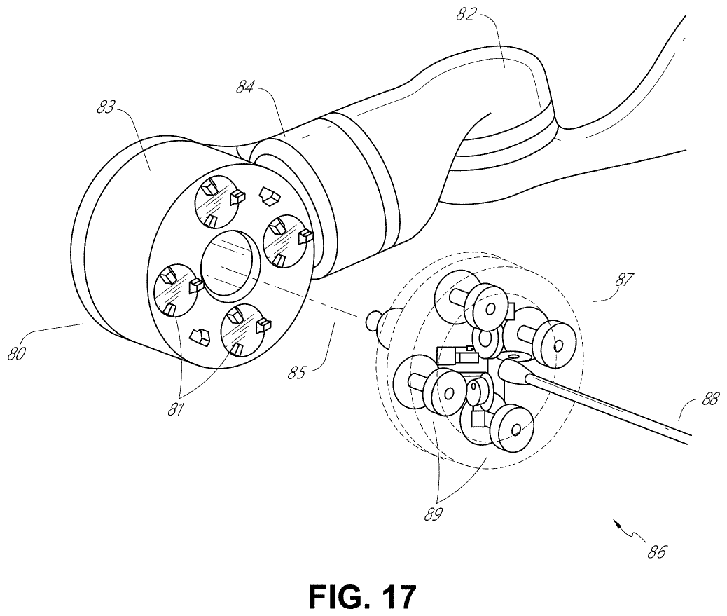

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver 80. Power and controls signals may be communicated from the non-rotational portion 84 of the instrument driver 80 to the rotational assembly 83 through electrical contacts that may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, the instrument shaft 88 extends from the center of the instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

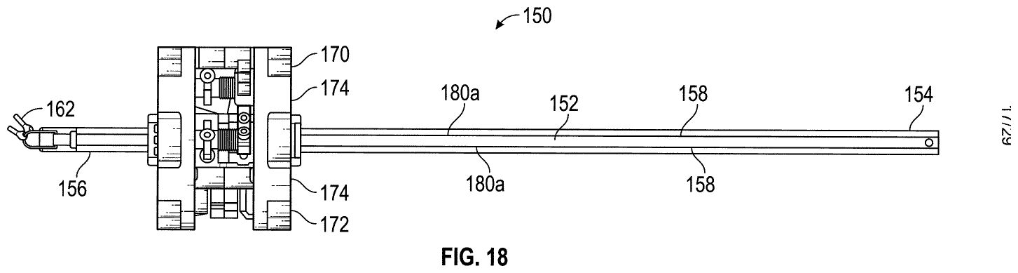

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver. In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

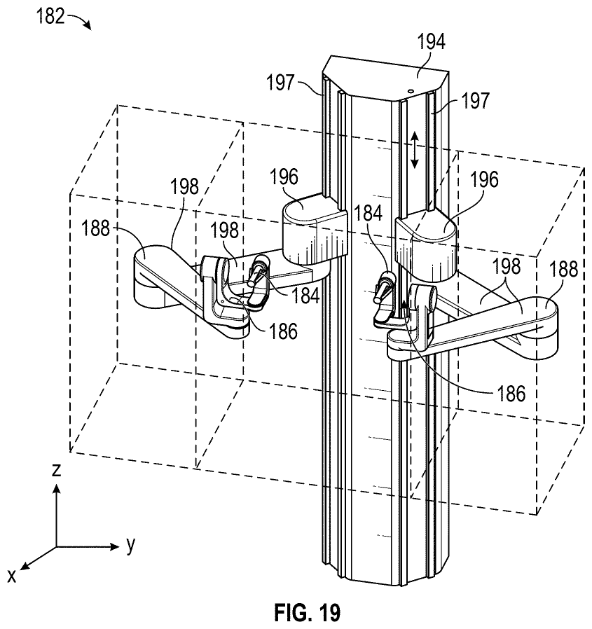

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as preoperative mapping, computer vision, real-time EM tracking, and robot command data may be used individually or in combination to achieve a radiation-free operating environment. In other cases, where radiation-based imaging modalities are still used, the preoperative mapping, computer vision, real-time EM tracking, and robot command data may be used individually or in combination to improve upon the information obtained solely through radiation-based imaging modalities.

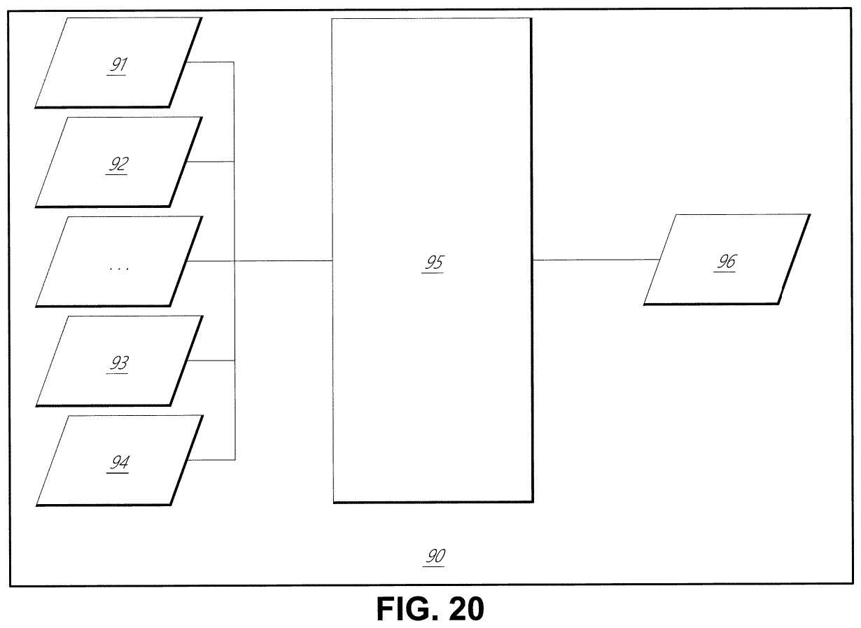

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart 11 shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Preoperative mapping may be accomplished through the use of the collection of low dose CT scans. Preoperative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data (or image data) 92. The localization module 95 may process the vision data 92 to enable one or more vision-based (or image-based) location tracking modules or features. For example, the preoperative model data 91 may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intraoperatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intraoperatively “registered” to the patient anatomy (e.g., the preoperative model) in order to determine the geometric transformation that aligns a single location in the coordinate system with a position in the preoperative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during preoperative calibration. Intraoperatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 20 shows, a number of other input data can be used by the localization module 95. For example, although not shown in FIG. 20, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. Specimen Collectors for Robotic Medical Systems

Robotic medical systems, such as those described above with reference to FIGS. 1-20 and others, can be used for robotic medical procedures that involve removing an object, specimen, or sample from a patient. For example, a robotic medical system can be used to perform a kidney stone removal procedure. During a robotic kidney stone removal procedure a physician can operate various robotic medical instruments (such as, e.g., the endoscopes and laparoscopes described above) using a controller. The robotic medical instruments can be engaged with robotic manipulators (such as robotic arms, robotic drive devices, and robotic instrument drive mechanisms) that position and manipulate the instruments.

As an example, a robotic medical system can include three robotic arms configured for use during a ureteroscopic kidney stone removal procedure. A first robotic arm can operate and control (e.g., control articulation of) a robotic ureteroscope and basketing device. A distal drive device positioned on a second robotic arm can insert and retract the ureteroscope into and out of the patient. In some embodiments, a third robotic arm can optionally be used to control a percutaneously inserted robotic laparoscope (e.g., during percutaneously assisted ureteroscopy (PAU)). The physician can control the system to capture a kidney stone with the basketing device. Then, when the robotic ureteroscope is holding the stone, the robotic ureteroscope can be retracted to remove the stone from the patient. Once positioned outside of the patient's body, the basketing device can be opened to release the stone. The robotic ureteroscope can then be reinserted into the body to retrieve further stones, if necessary. Generally, the stones are retained in order to be analyzed after the procedure.

This disclosure relates to specimen collectors that are configured for use with robotic medical systems in order to facilitate robotic medical procedures that involve removing objects, specimens, or samples from a patient. The specimen collectors can be configured such that the robotic medical system can deposit specimens therein robotically, which can minimize manual or physical interaction. During manual object removal procedures (e.g., manual kidney stone removal), objects removed from a patient are manually deposited into specimen cups, which are generally physically held by the physician or other sterile users in the operating room. Use of such manual specimen cups can be disadvantageous for use with robotic medical systems because such cups would need to be held by a clinician or have a specially designed holder, increasing cost and/or reducing the roboticization.

As will be described in more detail below, the specimen collectors described herein can be specifically configured for use with robotic medical systems so as to facilitate and/or optimize robotic procedures. For example, the specimen collectors described herein can be integrated into and/or configured to be supported by components of the robotic medical systems at positions at which the robotically controlled medical instruments can quickly and efficiently deposit specimens therein. As an initial example, a specimen collector configured for use with a robotic medical system can be integrated into a sterile drape configured to cover various robotic components of the system. The specimen collector can be positioned on the drape such that it is advantageously positioned when the drape is installed. For example, the specimen collector can be positioned at a location that is directly below a robotically controlled basketing device when the basketing device is retracted out of the patient. At this position, the basketing device can simply open to drop a retrieved object into the specimen collector. The specimen collector can be configured with at least one porous portion that allows fluid to drain therethrough while retaining objects deposited therein. The specimen collector can be configured with an opening-retaining device (e.g., pliable wires or metal strips) that are configured to hold an opening of the specimen collector open such that there is a large area for the objects to be deposited into. Further, the specimen collector can be configured such that it can be removed (e.g., torn away from) the drape, so the objects can be easily sent for analysis. In some embodiments, inclusion of the specimen collector on the drape provides a cost effective solution that provides significant benefit.

These and other features will be described in more detail below with reference to the embodiments illustrated in the figures, which are intended to illustrate certain example features and aspects of the specimen collectors described herein. The illustrated embodiments are not intended to be limiting, and those of skill in the art, upon consideration of this disclosure, will appreciate that various modifications can be made which are within the scope of this disclosure.

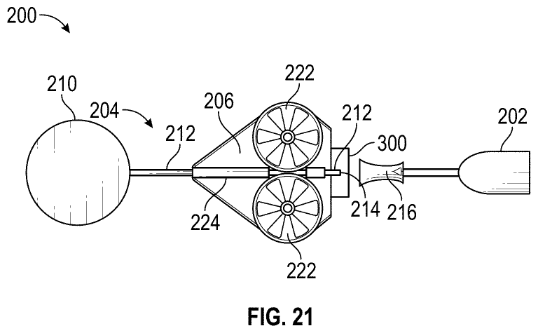

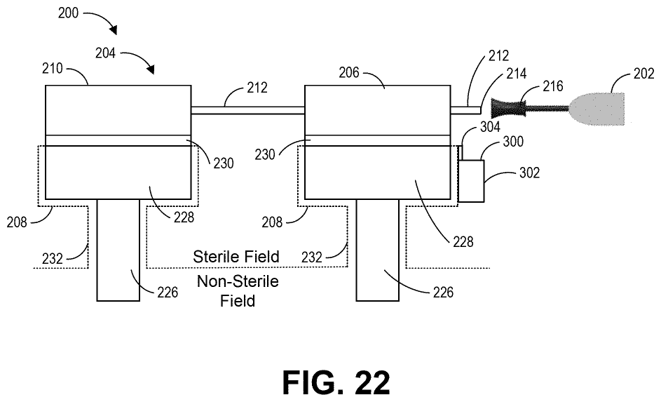

FIGS. 21 and 22 illustrate top and side views, respectively, of an embodiment a robotic medical system 200 including a specimen collector 300. The specimen collector 300 is configured such that specimens retrieved from within a patient 202 can be deposited therein using the robotic system 200. In the illustrated embodiment, the system 200 comprises, among other components, a medical instrument 204, a robotic manipulator (e.g., a drive device 206), a sterile barrier 208 (as shown in FIG. 22), and the specimen collector 300.

In the illustrated embodiment, the medical instrument 204 comprises a robotically controllable ureteroscope, which can be similar to the ureteroscope 32 described above with reference to FIG. 3. In other embodiments, the medical instrument 204 can comprise other types of medical instruments, such as any of the medical instruments described above with reference to FIGS. 1-20 as well as others, including endoscopes, laparoscopes, and catheters. As shown in the embodiment of FIGS. 21 and 22, the medical instrument 204 can include an instrument base 210 and an elongated shaft 212. For the illustrated embodiment, a proximal end of the elongated shaft 212 extends from the instrument base 210. In some embodiments, the elongated shaft 212 comprises a flexible shaft and/or an articulating shaft.

A distal end 214 of the elongated shaft 212 is configured to be inserted into the patient 202. For example, the robotic manipulator (e.g., the drive device 206 described in more detail below) can be configured to drive insertion and/or retraction of the elongated shaft 212 such that the distal end 214 of the elongated shaft 212 can be inserted into and retracted from the patient 202. In the illustrated embodiment, the medical instrument 204 is illustrated in a position wherein the distal end 214 of the elongated shaft 212 has been retracted out of the patient 202.

In the illustrated embodiment, the system 200 includes an access sheath 216. The access sheath 216 can be inserted into the patient 202 to provide a channel or conduit through which the elongated shaft 212 of the medical instrument 204 can be inserted. In the illustrated embodiment, the access sheath 216 is a ureteral access sheath inserted into the urethra of the patient 202, although other types of access sheaths, which can be inserted into other natural patient orifices or other surgical ports (e.g., laparoscopic ports) can also be used. In some embodiments, the access sheath 216 comprises a tube.

The medical instrument 204 can be configured to capture (e.g., grip, grab, hold, etc.) a specimen from within the patient. For example, in the case of a kidney stone removal procedure, the medical instrument 204 can include a basketing device configured to capture kidney stones such that the kidney stones can be removed from the patient 202. As described above, in some embodiments, the basketing device can be configured as a tool (robotically and/or manually controllable) which can be inserted through a working channel of the elongated shaft 212 of the medical instrument 204. In some embodiments, the basketing device is directly integrated into the medical instrument 204. Although examples described herein relate to kidney stone removal, the medical instrument 204 can be configured to collect and retrieve other types of objects, specimens, or samples from within the patient 202. For example, in some embodiments, the medical instrument 204 is configured to take a biopsy sample from the patient 202.

In the robotic medical system 200 illustrated in FIGS. 21 and 22, various robotic manipulators are shown for operating the robotic instrument 204. In the illustrated embodiment, the system 200 includes robotic manipulators configured as a drive device 206, instrument drive mechanisms 228, and robotic arms 226. The instrument drive mechanisms 228 and robotic arms 226 can be seen in the side view of FIG. 22. These robotic manipulators can be engaged with the medical instrument 204 in various ways as previously described and as will be described in more detail below.

In the illustrated embodiment, the drive device 206 is engaged with the elongated shaft 212 of the medical instrument 204 and configured to drive axial motion (e.g., insertion and/or retraction) of the distal tip 214 of the elongated shaft 212 into and out of the patient 202. For example, as shown in FIG. 21, the drive device 206 includes rollers 222 which can engage or contact the elongated shaft 212. In some embodiments, the rollers 222 can comprise a deformable material that provides grip between the rollers 222 and the elongated shaft 212. In some embodiments, the material comprises silicone rubber. In the illustrated embodiment, as the rollers 222 rotate, the elongated shaft 212 can be pulled, pushed, or otherwise driven axially through or relative to the drive device 206. Rotating the rollers 222 in a first direction can cause insertion of the elongated shaft 212, and rotating the rollers 222 in a second opposite direction can cause retraction of the elongated shaft 212. In some embodiments, other drive mechanisms can be used in place of or in addition to the rollers 222. In the illustrated embodiment, the elongated shaft 212 passes through a channel 224 of the drive device 206. The channel 224 can comprise a closed and/or an open channel. Use of an open channel 224 can facilitate loading the elongated shaft 212 of the medical instrument 204 into the drive device 206, which can simplify use of the device and decrease operating times. For example, the open channel can facilitate loading and/or unloading of the medical instrument 204 intraoperatively, or during a medical procedure, to allow a user such as a medical practitioner to manually make adjustments to the medical instrument 204, without having to fully retract the medical instrument 204 from within the patient.

As shown in FIG. 22, the drive device 206 can be attached, mounted, or otherwise connected to a robotic arm 226. The robotic arm 226 can include an instrument drive mechanism 228, and the drive device 206 can be attached to the instrument drive mechanism 228. The instrument drive mechanism 228 can include drive outputs configured to engage and actuate corresponding drive inputs on the drive device 206 to actuate the drive device 206. In the illustrated embodiment, a sterile adapter 230 is positioned between the instrument drive mechanism 228 and the drive device 206 such that the drive device 206 engages the instrument drive mechanism 228 through the sterile adapter 230. The sterile adapter 230 can be configured to link or otherwise transfer motion between the drive outputs of the instrument drive mechanism 228 and the corresponding drive inputs on the drive device 206. Further, the sterile adapter 230 can be a configured to define or provide a portion of the sterile barrier 208 (as best seen in FIG. 22) as described in more detail below.

With continued reference to FIG. 22, the robotic arm 226 to which the drive device 206 is mounted can be configured to move to manipulate the position of the drive device 206 in space. In some embodiments, for example, as illustrated, the drive device 206 can be positioned in proximity to the access sheath 216. Positioning the drive device 206 in proximity to the point at which the elongated shaft 212 will be inserted (e.g., close to the access sheath 216) can reduce buckling of the elongated shaft 212.

While the illustrated embodiment of the system 200 includes the drive device 206 for driving axial motion of the elongated shaft 212 of the medical instrument 204, other types of robotic manipulators can be used to drive axial motion in other embodiments. For example, in some embodiments, axial motion is driven by moving a robotic arm 226 to which the base 210 of the medical instrument 204 is attached. In other embodiments, the base 210 of the medical instrument 204 is configured to drive axial motion of the elongated shaft, for example, as described above with reference to FIG. 18.

As shown in FIG. 22, the base 210 of the medical instrument 204 can also be engaged with a robotic manipulator. In the illustrated embodiment, the base 210 is engaged with a second instrument drive mechanism 228 that is positioned on a second robotic arm 226. As illustrated, another sterile adapter 230 can be positioned between the instrument base 210 and the instrument drive mechanism 228. The instrument drive mechanism 228 engaged with the base 210 can be configured such that drive outputs of the instrument drive mechanism 228 drive corresponding drive inputs on the base 210 of the medical instrument 204 to control, for example, articulation of the elongated shaft 212 and/or articulation, opening, and/or closing of a basketing device. Engagement between the instrument base 210 and the instrument drive mechanism 228 is described above, for example, with reference to FIGS. 15-17.

The robotic arms 226 can be, for example, robotic arms mounted on or extending from a cart as shown in FIGS. 1-4 and/or robotic arms extending from a patient platform or table as shown in FIGS. 5-14. Example instrument drive mechanisms 228, which can be positioned on the distal ends of the robotic arms 226 are shown in FIGS. 16-18.

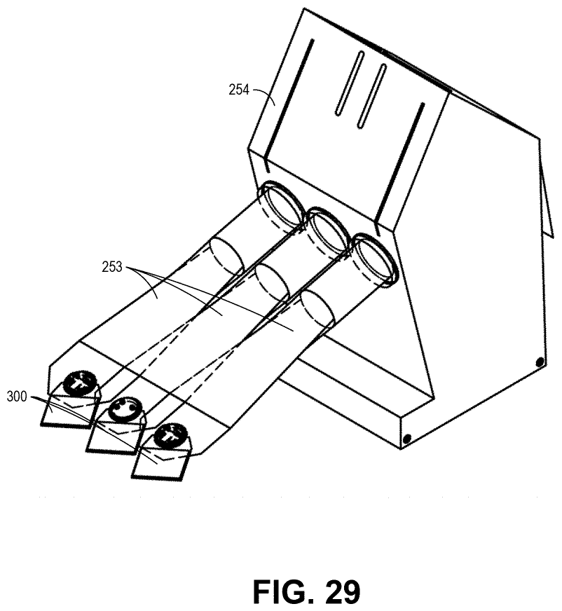

FIG. 22 illustrates that the robotic medical system 200 can include the sterile barrier 208. The sterile barrier 208 can be configured to separate a sterile field from a non-sterile field. In the illustrated embodiment, the sterile barrier 208 is provided by one or more sterile drapes 232 and the sterile adapters 230 previously described. The sterile drapes 232 can comprise flexible sheets (e.g., plastic sheets) configured in size and shape to cover components of the robotic medical system 200 that are positioned within the non-sterile field. As illustrated, the sterile drapes 232 cover the robotic arms 226 and instrument drive mechanisms 228. More detailed examples of sterile drapes are shown in FIGS. 29-31 which are described in more detail below.

As shown in FIG. 22, some components of the robotic medical system 200 are positioned within the sterile field and other components are positioned within the non-sterile field. For example, in the illustrated embodiment, the medical instrument 204, drive device 206, access sheath 216, and the patient 202 are positioned within the sterile field, while the robotic arms 226 and instrument drive mechanisms 228 are positioned in the non-sterile field. Other configurations are also possible.

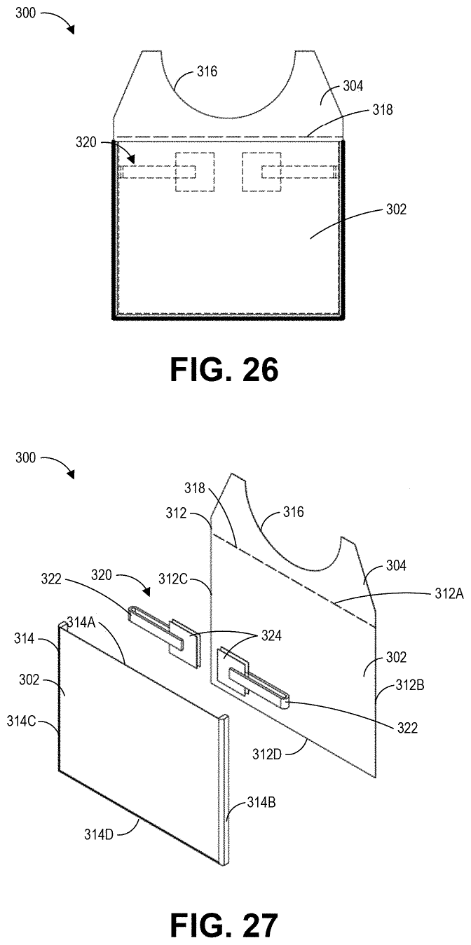

As noted above, the system 200 also includes the specimen collector 300 into which specimens removed from the patient 202 using the medical instrument 204 can be deposited. As best seen in the side view of FIG. 22, the specimen collector 300 can include a receptacle portion 302 and a connector 304. The receptacle portion 302 is configured to provide a receptacle, container, vessel, or repository into which specimens can be deposited, and the connector 304 is configured to attach the receptacle portion 302 to a component of the robotic medical system 200 to support and position the specimen collector 300. The connector 304 can be coupled to the receptacle portion 302. In some embodiments, the receptacle portion 302 is made from a flexible material, such as a sheet of plastic formed so as to create a receptacle or container. In some embodiments, the receptacle portion 302 comprises a flexible bag. The receptacle portion 302 includes an opening through which specimens can be deposited therein. In some embodiments, the specimen collector 300 further comprises an open-retaining device positioned the opening of the receptacle portion 302 and configured to retain the opening in an open configuration to facilitate depositing specimens therein. Various features and embodiments of the receptacle portion 302 and the connector 304 will be described in more detail below. A more detailed embodiment of the specimen collector 300 is shown in FIGS. 26-27.

As shown in FIG. 22, the specimen collector 300 can be positioned within the sterile field. For example, in the illustrated embodiment, the connector 304 of the specimen collector 300 is attached to the sterile drape 232 which covers the robotic arm 226 to which the drive device 206 is connected. Other locations for the specimen collector 300 are also possible. For example, in some embodiments, the connector 304 attaches the specimen collector 300 to the sterile adapter 230, to the access sheath 216, or to the drive device 206 or instrument drive mechanism 228 itself.

FIGS. 21 and 22 also illustrate that, in some embodiments, the specimen collector 300 can be advantageously positioned on the robotic system 200 so as to facilitate depositing specimens therein. In the illustrated embodiment, the specimen collector 300 is positioned at a location that is below (e.g., directly below) the distal tip 214 of the elongated shaft 212 of the medical instrument 204 when the distal tip 214 is withdrawn from the patient 202 and/or the access sheath 216. In this position, depositing the specimen into the receptacle portion 302 can be accomplished by releasing the specimen (e.g., opening the basketing device) and allowing the specimen to fall into the receptacle portion 302 due to gravity. This position for the specimen collector 300 can also maintain alignment between the elongated shaft 212 and the access sheath 216 such that, after the specimen is deposited into the receptacle portion 302, the distal tip 214 of the elongated shaft 212 can be quickly reinserted into the patient 202 to continue the procedure. This can reduce the overall length of the procedure, improving patient outcomes.

Further, as illustrated in FIGS. 21 and 22, in some embodiments, the specimen collector 300 can be advantageously positioned on the robotic system 200 at a location that is in proximity to the access into the patient (e.g., the access sheath 216). In the illustrated embodiment, for example, the specimen collector 300 is positioned on the distal (i.e., patient facing) side of the drive device 206. This position can beneficially minimize the amount of movement required to position the distal tip 214 of the medical instrument 204 over the specimen collector 300. Again, this can reduce the overall length of the procedure. Additionally, the robotic arm 226 can position the drive device 206 in proximity the access into the patient 202 to further minimize the amount of movement required to position the distal tip 214 of the medical instrument 204 over the specimen collector 300. For example, as shown in FIG. 22, the distal side of the drive device 206 is located in proximity to the proximal side of the access sheath 216 such that the specimen collector is positioned just proximally of the access sheath 216.

As shown in FIG. 22 (and as will be described in more detail below with reference to FIGS. 26 and 27), the connector 304 can comprise an attachment tab. The receptacle portion 302 can extend from the attachment tab. That is, the receptacle portion 302 can be attached to the attachment tab. The attachment tab can be configured to attach to the component of the robotic medical system 200 that supports the specimen collector 300. For example, the attachment tab can be configured to attach to the sterile drape 232, the sterile adapter 230, the drive device 206, the access sheath 216, or other components of the robotic medical system 200.

As noted above, in the illustrated embodiment, the connector 304 is attached to the sterile drape 232. In some embodiments, the connector 304 is fixedly or permanently attached to the sterile drape 232. That is, in some embodiments, the specimen collector 300 is a component of the sterile drape 232. In these embodiments, the specimen collector 300 can positioned on the drape 232 such that, when the drape 232 is installed over the robotic medical system 200, the specimen collector 300 is positioned in an advantageous or desired position as described above. In other embodiments, the connector 304 can be configured to selectively attach to the sterile drape 232 (or other components of the robotic medical system 200). For example, the connector 304 can comprise an adhesive strip on the attachment tab. A user can then use the adhesive strip to attach the specimen collector 300 to a component of the robotic medical system 200 as desired.

The receptacle portion 302 can be attached to the attachment tab or connector 304 in a removable manner such that the receptacle portion 302 can be removed from the connector 304. In some embodiments, once specimens are deposited into the receptacle portion 302, the receptacle portion 302 can be removed from the connector 304 while retaining the specimens therein. The receptacle portion 302 can then be sent for analysis of the specimens. As will be described below, in some embodiments, the specimen collector 300 comprises a perforation between the attachment tab or connector 304 and the receptacle portion 302 configured such that the receptacle portion 302 can be torn from the attachment tab or connector 304. Other methods for removing the receptacle portion 302 from the connector 304 are also possible as described below.

In some embodiments, at least a portion of the receptacle portion 302 is porous and configured to allow fluid to drain from the receptacle portion 302 while retaining the specimen. During some medical procedures, a fluid, such as an irrigant used during the procedure or a patient fluid, can get into the receptacle portion 302. The porous portion of the receptacle portion 302 can allow this fluid to drain. In some embodiments, the specimen collector 300 can include a drainage port that can be connected to a fluidics system which can actively or passively collect such fluid from the receptacle portion 302. The porosity of the porous portion can be configure such that fluid drains therethrough, while collected specimens are retained within the receptacle portion.

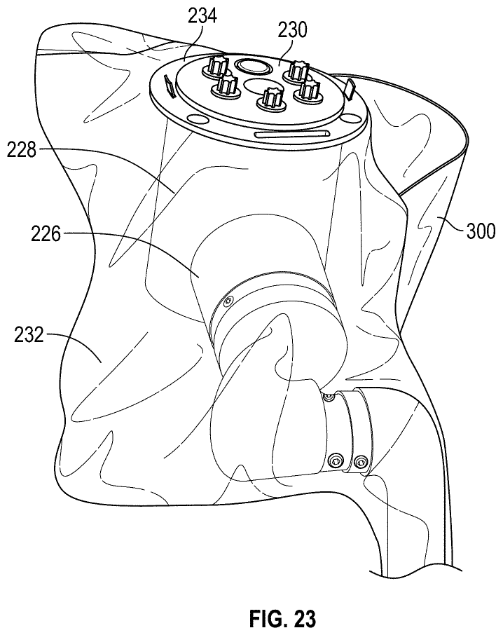

FIG. 23 is a perspective view of a distal end of the robotic arm 226 with the instrument drive mechanism 228 positioned thereon. In the illustrated embodiment, the robotic arm 226 is covered with a sterile drape 232. As shown, the sterile drape 232 can be part of a sterile barrier that includes a sterile adapter 230 configured to fit over the instrument drive mechanism 228. The sterile adapter 230 can have a collar 234 that can be configured to engage with the sterile drape 232, and the sterile adapter 230 can provide a sterile interface between the instrument drive mechanism 228 and a component attached thereto (such as an instrument base 210 or drive device 206).

FIG. 23 also illustrates an embodiment of a specimen collector 300 attached to the sterile drape 232. In some embodiments, the specimen collector 300 can be attached to the sterile drape 232 at the collar 234. In the embodiment illustrated in FIG. 23, the specimen collector 300 is configured as a flexible bag.

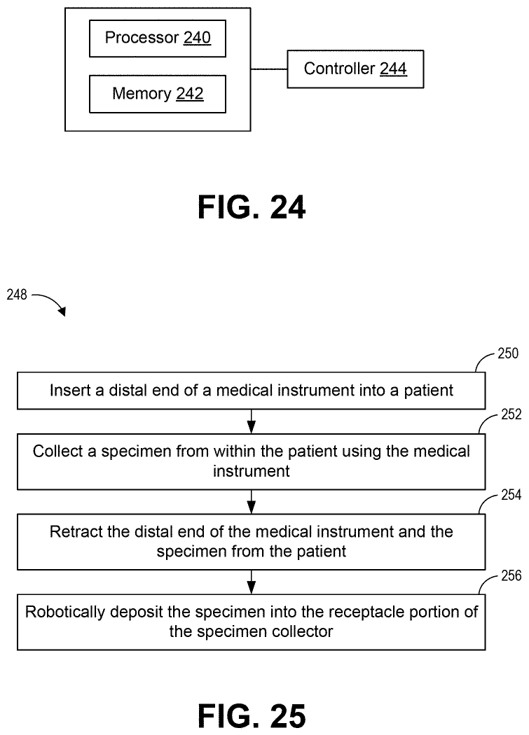

FIG. 24 is a block diagram of example control components for the robotic medical system 200. In the illustrated embodiment, the control components include a processor 240, a memory 242, and a controller 244. The memory 242 can include instructions that configure the processor 240 to execute various functions to control aspects of the robotic medical system 200. For example, the memory 242 can include instructions that, when executed, configure the processor 240 to perform the functions described below with reference to FIG. 25. A physician or other operator can use the controller 244 to provide inputs for controlling the robotic medical system 200. In some embodiments, the controller 244 is a handheld controller including one or more joysticks, buttons, or other users inputs. In some embodiments, the controller 244 can be the controller described above with reference to FIG. 19.

FIG. 25 is a flow chart illustrating an example control method 248 that can be executed using the control components of FIG. 24 to operate the robotic medical system 200. The control method can be stored, for example, as instructions in the memory 242. The method 248 can begin at block 250, at which the instructions configure the processor 240 to control insertion of the distal end 214 of the medical instrument 204 into the patient 202. In some embodiments, insertion is commanded and/or otherwise controlled by the physician using the controller 244. As noted above, insertion can be provided in a variety of ways. For example, with reference to the embodiment illustrated in FIGS. 21 and 22, the drive device 206 can drive insertion using the rollers 222. In other embodiments, insertion can be achieved by moving the medical instrument 204 using the robotic arm 226 and/or by driving insertion of the elongated shaft 212 relative to the instrument base 210 using an instrument based insertion architecture, for example, as described with reference to FIG. 18. In some embodiments, insertion is provided through the access sheath 216.

At block 252, the method 248 can include collecting the specimen from within the patient 202 using the medical instrument 204. In some embodiments, the physician can navigate and control the distal tip 214 of the medical instrument 204 within the patient 202 using the controller 244, allowing the physician to locate and collect the sample. As noted above, in the case of a kidney stone removal procedure, collecting the sample can comprise capturing a kidney stone within a basketing device inserted through a working channel of the elongated shaft 212 of the medical instrument 204.

With the sample collected, the method 248 moves to block 254, at which the distal end 214 and collected sample is retracted from the patient 202. Retraction can be commanded, for example, by the physician using the controller 244. Retraction can be provided using the same mechanisms as described above with regards to insertion. For example, retraction can be driven by the drive device 206, by moving a robotic arm 226, and/or with an instrument-based insertion architecture that drives retraction of the elongated shaft 212 relative to the instrument base 210. At block 254, the distal tip 214 of the medical instrument 204 can be retracted to a position at which the specimen can be deposited into the specimen collector 300. For example, the distal tip 214 can be retracted to a position above the specimen collector 300 as shown in FIGS. 21 and 22. In some embodiments, retraction to this depositing position can be triggered by a single user command. For example, once the specimen is captured, the user can provide a single input on the controller 244 which can cause the system 200 to automatically retract the distal tip 214 to the depositing position.

At block 256, the method 248 can include depositing the specimen into the receptacle portion 302 of the specimen collector 300. In the illustrated embodiment of the system 200, depositing the specimen into the receptacle portion 302 of the specimen collector 300 can include releasing the specimen from the distal tip 214 of the medical instrument 204 such that the specimen falls under the force of gravity into the receptacle portion 302 of the specimen collector 300. In other embodiments, depositing can be accomplished by articulating the elongated shaft 212 of the medical instrument 204 to insert the specimen into the specimen collector 300. In some embodiments, depositing the specimen into the specimen collector 300 can occur automatically after receipt of a deposit command provided with the controller 244. For example, upon receipt of the command, the system 200 can automatically move the distal end 214 of the medical instrument 204 into a depositing position and robotically deposit the specimen into the receptacle portion 302 of the specimen collector 300. In some embodiments, the system 200 is aware of the location of the specimen collector 300 such that movement to and depositing into the specimen collector 300 can be automatically performed by the system 200 (e.g., automatically performed upon receipt of a user command). That is, in some embodiments, the physician need not navigate the distal end 214 to the specimen collector 300; instead, such navigation can occur automatically.

FIGS. 26 and 27 are a front and perspective exploded views, respectively, of an embodiment of a specimen collector 300. In the illustrated embodiment, the specimen collector 300 comprises a receptacle portion 302 configured to receive a specimen removed from the patient and a connector 304 coupled to the receptacle portion 302 and configured to attach to a medical system (e.g., to a component thereof, such as a drape) so as to position the receptacle portion 302 relative to the medical system. A porous portion can be included at least a portion of the receptacle portion 302. The porous portion can be configured to allow liquid to drain from the receptacle portion 302 while retaining a specimen deposited within the receptacle portion 302.

As best shown in the exploded view of FIG. 27, the specimen collector 300 can comprise a first layer 312 and a second layer 314. In some embodiments, each of the first layer 312 and the second layer 314 can comprise a flexible layer, such as a plastic sheet, such that the specimen collector 300 comprises a flexible bag-like structure. The receptacle portion 302 can be formed between the first layer 312 and the second layer 314. For example, the first layer 312 can comprise a first upper edge 312A, a first right edge 312B, a first left edge 312C, and a first lower edge 312D, and the second layer 314 can include comprise a second upper edge 314A, a second right edge 314B, a second left edge 314C, and a second lower edge 314D. The second right edge 314B, the second left edge 314C, and the second lower edge 314D can be connected to the first right edge 312B, the first left edge 312C, and the first lower edge 312D, respectively, such that the first flexible layer 312 and the second flexible layer 314 form a pocket having an opening defined by (e.g., between) the first upper edge 312A and the second upper edge 314A.

The connector 304 can comprise an attachment tab formed by a portion of the first layer 312 that extends from the first upper edge 312A. As shown in FIGS. 26 and 27, the connector 304 can include a cutout 316. The cutout 316 can be configured in size and shape to correspond to a component to which the connector 304 can attach. For example, in the illustrated embodiment, the cutout 316 is semi-circular so as to correspond to the generally circular shape of the collar 234 (FIG. 23) and/or instrument drive mechanism 228 or sterile adapter 230 (FIGS. 21 and 22). Other shapes and configurations for the cutout 316 on the connector 304 are also possible. In some embodiments, the cutout 316 can be omitted.

In some embodiments, the connector 304 or attachment tab can be permanently connected to another structure, such as the sterile drape 232 or sterile adapter 230, such that the specimen collector 300 is a component of that structure. The connector 304 can be connected to that structure such that the specimen collector 300 is advantageously positioned at a desired location when that structure is installed on the robotic system. In other embodiments, the connector 304 or attachment tab is configured to selectively attach to the robotic system. For example, the connector 304 or attachment tab can include an adhesive backing on at least a first side of thereof such that the specimen collector 300 can be adhesively attached to the robotic medical system 200 at a desired location.

As shown in FIGS. 26 and 27, on the first layer 312, the connector 304 can be attached to the receptacle portion 302 with a perforated portion 318. That is, a perforation 318 can be positioned between the receptacle portion 302 and the connector 306. The perforation can be configured such that the receptacle portion 302 can be torn away from the connector 306 as described above. In some embodiments, other methods for configuring the receptacle portion 302 to be removable from the connector 304 are possible. For example, the perforation 318 can be replaced with a tear strip or other suitable structure.

The specimen collector 300 can also include an open-retaining device 320. The open-retaining device 320 can be configured to retain the opening of the receptacle portion 302 in an open configuration so as to facilitate depositing of specimens into the receptacle portion 302. In some embodiments, for example, as illustrated, the open-retaining device 320 can be positioned at the opening of the receptacle portion 302. In the illustrated embodiment, the open-retaining device 320 comprises a formable metal strips 322. In the illustrated embodiment, the formable metal strips 322 are attached on a first side to the first layer 312 with an attachment pad 324 and attached on a second side to the second layer 314 with an attachment pad 324. The formable metal strips 322 can be bent into a configuration that holds the opening of the receptacle portion 302 open. Other mechanisms for the open-retaining device 320 are also possible, such as formable, shape retaining wires that can be embedded in the opening. In some embodiments, the open-retaining device 320 can be omitted.

As noted above, the specimen collector 300 can include a porous portion for allowing fluid to drain from the receptacle portion 302. In some embodiments, one or both of the first layer 312 and the second layer 314 can be porous. In some embodiments, a portion of one or both of the first layer 312 and the second layer 314 can be porous.

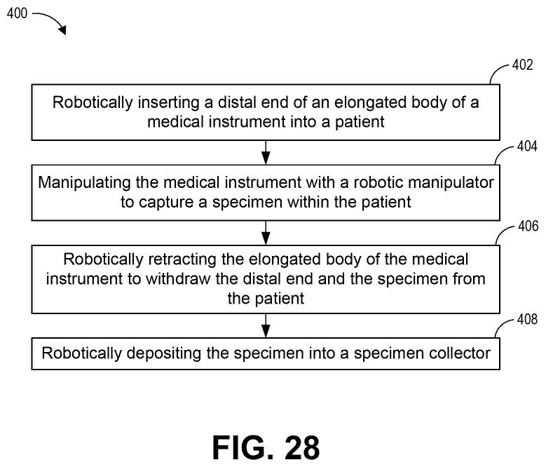

FIG. 28 is a flowchart illustrating an example method 400 for using a robotic medical system, such as the robotic medical system 200, to deposit a specimen in a specimen collector 300. The method 400 can begin at block 402, which includes robotically inserting the distal end 214 of the elongated body 212 of the medical instrument 204 into the patient 202. In some embodiments, the robotic manipulator comprises the drive device 206 described above that is configured to engage with and drive insertion and retraction of the elongated body of the medical instrument. Insertion can be driven by the drive device 206. In some embodiments, the robotic manipulator comprises a robotic arm 226 and an instrument drive mechanism 228 positioned at a distal end of the robotic arm 226. The instrument drive mechanism 228 can be configured to attach to the base 210 of the medical instrument 204 to operate the medical instrument 204. In some embodiments, robotically inserting the elongated body 212 of a medical instrument comprises moving the robotic arm 226. In some embodiments, robotically inserting the elongated body 212 of the medical instrument 204 comprises driving an insertion mechanism of the base 210 with the instrument drive mechanism 228 to cause the elongated body 212 to be inserted relative to the base 210.

At block 404, the method 400 includes manipulating the medical instrument 204 with a robotic manipulator to capture a specimen within the patient. In some embodiments, the base 210 of the medical instrument 204 is engaged with the instrument drive mechanism 228 such that drive outputs of the instrument drive mechanism 228 actuate drive inputs in the base 210 to cause articulation of the elongated shaft 212. The physician or other operator can control articulation and/or insertion and retraction of the elongated shaft 212 to capture the specimen with the distal end 214 of the medical instrument 204. Navigation within the patient can be facilitated by the navigation and localization system described above with reference to FIG. 20.

At block 406, the method 400 includes robotically retracting the elongated shaft 212 of the medical instrument 204 to withdraw the distal end 214 and the specimen from the patient. In some embodiments, the robotic manipulator comprises the drive device 206 configured to engage with and drive insertion and retraction of the elongated shaft 212 of the medical instrument 204. Retraction can be driven by the drive device 206. In some embodiments, the robotic manipulator comprises a robotic arm 226 and an instrument drive mechanism 228 positioned at a distal end of the robotic arm 226. The instrument drive mechanism 228 can be configured to attach to the base 210 of the medical instrument 204 to operate the medical instrument 204. In some embodiments, robotically retracting the elongated shaft 212 of a medical instrument 204 comprises moving the robotic arm 226. In some embodiments, robotically retracting the elongated body 212 of the medical instrument 204 comprises driving a retraction mechanism of the base 210 with the instrument drive mechanism 228 to cause the elongated shaft 212 to be retracted relative to the base 210.

At block 408, the method 400 includes robotically depositing the specimen into the specimen collector 300. As described with reference to FIGS. 21 and 22, depositing the specimen into the specimen collector 300 can comprise positioning the specimen over the specimen collector 300 and releasing the specimen such that it falls into the specimen collector 300. In some embodiments, robotically depositing the specimen into the specimen collector comprises, upon receipt of a user command, automatically moving the distal end 214 of the elongated shaft 212 into a depositing position relative to the specimen collector 300.

The method 400 may also include draping a robotic medical system with a sterile barrier 232 to separate a sterile field containing at least the medical instrument 204 and the specimen collector 300 from a non-sterile field containing at least the robotic manipulator. In some embodiments, the specimen collector 300 is attached to the sterile barrier 232 at a location at which the medical instrument can robotically deposit the specimen.

In some embodiments, the method 400 also includes adhesively attaching the specimen collector 300 to the sterile barrier 232. In some embodiments, the receptacle portion 302 of the specimen collector 300 is removable from the connector 304, and the method 400 further comprises detaching the receptacle portion 302 from the connector 304. The method 400 may further include draining fluid through a porous portion of the receptacle portion 302 while retaining the specimen within the receptacle portion 302. In some embodiments, the method 400 also includes positioning an opening of the specimen collector in an open position using an open-retaining device 320 of the specimen collector 300.

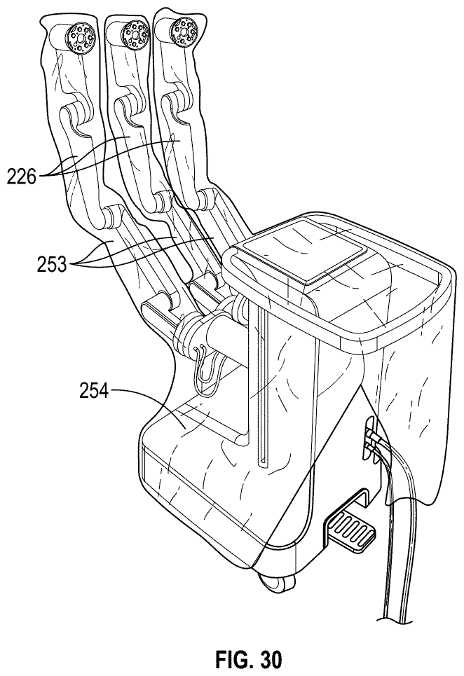

FIG. 29 illustrates an isometric view of an embodiment of a sterile drape 232 including specimen collectors 300. The sterile drape 232 can form part of the sterile barrier 208 that can also include sterile adapters 230 as shown in FIGS. 21 and 22. The sterile drape 232 can be made from a sterile, flexible material, such a plastic sheeting. In the illustrated embodiment, the sterile drape 232 is configured to drape a cart of a robotic medical system that includes three robotic arms, for example, as shown in FIGS. 2 and 30. As shown, the sterile drape 232 includes three flexible tubes 253. The three flexible tubes 253 are configured in size and shape to be fitted over three robotic arms. The flexible tubes 253 extend from a cart cover portion 254 configured in size and shape to be fitted over a cart.

In the illustrated embodiment, specimen collectors 300 are positioned at the distal ends of each of the flexible tubes 253 so as to be positioned in an advantageous position (as described above) when the drape 232 is installed. FIG. 30 is a perspective view of an embodiment of the sterile drape 232 of installed on a cart including three robotic arms 226. In the illustrated embodiment, the robotic arms 226 have been moved into an example draping position that can facilitate installation of the drape 232. Once the drape 232 is installed, the robotic arms 226 can then move into position for a medical procedure.

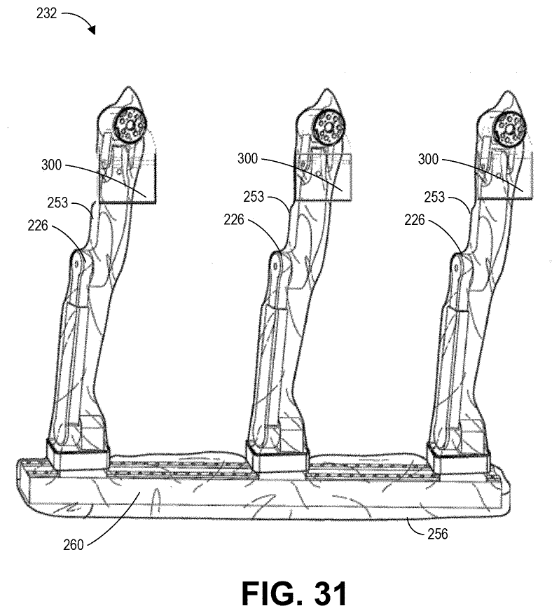

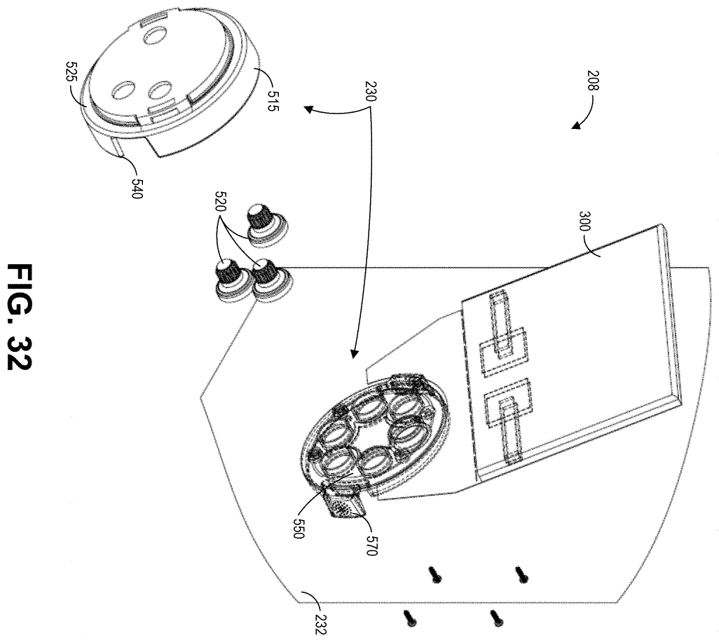

FIG. 31 illustrates another embodiment of a sterile drape 232 including specimen collectors 300. The drape 232 of FIG. 31 is configured for use with a robotic medical system that includes robotic arms 226 that are movably mounted on a bar or rail 260. Such a system is shown and described above with reference to FIGS. 12-14. In this embodiment, the drape 232 includes three flexible tubes 253 configured to drape the robotic arms 226 and a rail drape portion 256 configured to drape the rail 260. As shown, specimen collectors 300 can be positioned at the distal ends of the flexible tubes 253.

FIG. 32 illustrates an embodiment of the sterile barrier 208 configured as an assembly including the sterile drape 232, the sterile adapter 230, and specimen collector 300. The sterile adapter 230 includes an upper plate 540, a lower plate 550, and torque couplers 520 rotatably supported in the sterile adapter 230 so that they are rotatable about their respective drive axes with respect to the upper and lower plates 540, 550. The sterile adapter 230 includes an attachment mechanism 570 (e.g., a clip, latch, magnet, etc.), that can secure the sterile adapter 230 to the instrument drive mechanism 228. The attachment mechanism 570 and/or torque couplers 520 in the sterile adapter 230 can align with corresponding features on the instrument drive mechanism 228, and the specimen collector 300 can be attached to the sterile barrier 208 in a known or fixed position with respect to the sterile adapter 230 so that the robotic arm can hold the specimen collector 300 in an advantageous position for specimen collection upon securing the sterile barrier to the robotic system, without a need for the user to manually position the specimen collector 300. For example, as shown in FIG. 32, the sterile adapter can define a distal side 515 and a proximal side 525 based on the positions of the set of torque couplers 520 and the attachment mechanism 570. The specimen collector 300 can be attached to the sterile on the distal side 515 of the sterile adapter 230. With such a position, a medical instrument (e.g., a ureteroscope and basketing tool) extending from the distal side can readily deposit the specimen into the specimen collector on the distal side 515 upon extracting the specimen and retracting out of the patient's body.

### 3. Implementing Systems and Terminology

Implementations disclosed herein provide systems, methods and apparatus for specimen collectors configured for use with robotic medical systems.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The phrases referencing specific computer-implemented processes/functions described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

What is claimed is:

1. A robotic medical system, comprising:
a medical instrument comprising a distal end configured to be inserted into a patient and capture a specimen within the patient;
a robotic manipulator engaged with the medical instrument and configured to operate the medical instrument;
a sterile barrier configured to separate a sterile field containing the medical instrument from a non-sterile field containing the robotic manipulator; and
a specimen collector comprising:
a receptacle portion in the sterile field and configured to receive the specimen when the distal end of the medical instrument is withdrawn from the patient, and
a connector coupled to the receptacle portion and configured to attach to the robotic medical system.

2. The system of claim 1, further comprising at least one processor configured, via operation of the robotic manipulator, to:
insert the distal end of the medical instrument into the patient;
collect the specimen from within the patient using the medical instrument;
retract the distal end of the medical instrument and the specimen from the patient; and
robotically deposit the specimen into the receptacle portion of the specimen collector.

3. The system of claim 2, wherein the at least one processor is further configured to, upon receipt of a user command, operate the robotic manipulator to:
move the distal end of the medical instrument into a depositing position; and
robotically deposit the specimen into the receptacle portion of the specimen collector.

4. The system of claim 1, wherein:
the connector comprises an attachment tab; and
the receptacle portion is removable from the attachment tab.

5. The system of claim 4, wherein the specimen collector comprises a perforation between the attachment tab and the receptacle portion configured such that the receptacle portion can be torn from the attachment tab.

6. The system of claim 1, wherein at least a portion of the receptacle portion is porous and configured to allow fluid to drain from the receptacle portion while retaining the specimen.

7. The system of claim 1, wherein the specimen collector further comprises an open-retaining device positioned at an opening of the receptacle portion and configured to retain the opening in an open configuration.

8. The system of claim 1, wherein the sterile barrier comprises a sterile drape configured to cover at least a portion of the robotic manipulator.

9. The system of claim 1, wherein:
the sterile barrier comprises a sterile adapter configured to be positioned between a base of the medical instrument and the robotic manipulator such that the robotic manipulator engages the medical instrument via the sterile adapter;
the sterile adapter defines a distal side and a proximal side such that the medical instrument extends from the distal side; and
the connector is attached to the sterile barrier on the distal side such that, when the sterile adapter is positioned on the robotic manipulator, the receptacle portion is positioned within the sterile field between the robotic manipulator and an access site of the patient.

10. The system of claim 1, wherein:
the connector comprises an attachment tab, and
the attachment tab comprises an adhesive surface configured to adhere to the robotic medical system such that the receptacle portion is positioned within the sterile field at a location at which the medical instrument can robotically deposit the specimen.

11. A robotic medical method, comprising:
robotically inserting a distal end of an elongated body of a medical instrument into a patient;
manipulating the medical instrument with a robotic manipulator to capture a specimen within the patient;
robotically retracting the elongated body of the medical instrument to withdraw the distal end and the specimen from the patient; and
robotically depositing the specimen into a specimen collector.

12. The method of claim 11, further comprising:
draping a robotic medical system with a sterile barrier to separate a sterile field containing at least the medical instrument and the specimen collector from a non-sterile field containing at least the robotic manipulator,
wherein the specimen collector is attached to the sterile barrier at a location at which the medical instrument can robotically deposit the specimen.

13. The method of claim 11, wherein robotically depositing the specimen into the specimen collector comprises, upon receipt of a user command:
automatically moving the distal end of the elongated into a depositing position relative to the specimen collector; and
automatically robotically depositing the specimen into the specimen collector.

14. The method of claim 11, wherein the specimen collector comprises:
a receptacle portion configured to receive the specimen removed from the patient with the medical instrument, and
a connector extending from the receptacle portion and attached to and supported by a component of a robotic medical system such that the receptacle portion is positioned at a location at which the medical instrument can robotically deposit the specimen.

15. The method of claim 14, further comprising draining fluid through a porous portion of the receptacle portion while retaining the specimen within the receptacle portion.

16. A specimen collector for a medical system, the specimen collector comprising:
a receptacle portion configured to receive a specimen removed from a patient;
a connector coupled to the receptacle portion and configured to attach to a medical system so as to position the receptacle portion relative to the medical system; and
a porous portion formed on at least a portion of the receptacle portion, the porous portion configured to allow liquid to drain from the receptacle while retaining a specimen deposited within the receptacle portion.

17. The specimen collector of claim 16, wherein the receptacle portion comprises:
a first flexible layer comprising a first upper edge, a first right edge, a first left edge, and a first lower edge, and wherein the connector extends from the first upper edge; and
a second flexible layer comprising a second upper edge, a second right edge, a second left edge, and a second lower edge, wherein the second right edge, the second left edge, and the second lower edge are connected to the first right edge, the first left edge, and the first lower edge, respectively, such that the first flexible layer and the second flexible layer form a pocket having an opening defined by the first upper edge and the second upper edge.

18. The specimen collector of claim 17, further comprising a perforation positioned between the receptacle portion and the connector and configured such that the receptacle portion can be torn away from the connector.

19. The specimen collector of claim 16, further comprising an open-retaining device positioned at an opening of the receptacle portion and configured to retain the opening in an open configuration.

20. The specimen collector of claim 16, wherein the connector comprises an attachment tab including an adhesive backing on at least a first side of the attachment tab.

21. A sterile barrier for a robotic medical system, the barrier comprising:
a sterile drape comprising a first flexible tube configured to cover at least a portion of a first robotic arm; and
the specimen collector of claim 16, wherein an attachment tab of the sample collector device is attached to the sterile drape at a distal end of the first flexible tube.

22. The barrier of claim 21, wherein the sterile drape further comprises:
a second flexible tube configured to cover a second robotic arm;
a third flexible tube configured to cover a third robotic arm; and
a flexible cart cover configured to drape a cart from which the first, second, and third robotic arms extend.

23. The barrier of claim 21, further comprising:
a sterile adapter connected to a distal end of the first flexible tube,
wherein the specimen collector is attached to the sterile barrier proximate the distal end of the first flexible tube.
